(19) United States
(12) Patent Application Publication
(10) Pub. No.: US 2003/0036691 A1
(43) Pub. Date: Feb. 20, 2003

(54) Capacitively coupled electrode system with variable capacitance for sensing potentials at the surface of tissue

(76) Inventors: Thomas G. Stanaland, Winchester, CA (US); Gaetan Chevalier, Encinitas, CA (US)

Correspondence Address: STRADLING YOCCA CARLSON & RAUTH, IP Department, P.O. Box 7680, 660 Newport Center Drive, Suite 1600, Newport Beach, CA, 92660-6441, US

(21) Appl. No.: 10/266,648

(22) Filed: Oct. 7, 2002

Related U.S. Application Data
(62) Division of application No. 09/636,541, filed on Aug. 10, 2000.

(51) Int. Cl. A61B 5/04

(52) U.S. Cl. 600/372

(57) ABSTRACT

An electrical activity sensor for sensing and reproducing electrical potentials at the surface of a test item such as a human being has an electrode configured to be capacitively coupled to the test item and a variable capacitance coupled to the electrode. The capacitively coupled electrode and the variable capacitance cooperate to mitigate a need for conductively coupling the electrode to the test subject.

### FIELD OF THE INVENTION

The present invention relates generally to medical electrical sensing devices such as electroencephalograph (EEG), electromyograph (EMG), electrocardiograph (EKG) and galvanic skin response (GRS) devices. The present invention relates more particularly to a capacitively coupled electrode system including a capacitively coupled electrode in electrical communication with a variable capacitance device for sensing and reproducing electric potentials generated at the surface of living tissue, as well as at the surface of any other test item.

### BACKGROUND OF THE INVENTION

The use of electrodes for sensing electrical activity at the surface of living tissue, such as during the performance of an electroencephalograph (EEG), an electromyograph (EMG), an electrocardiograph (EKG) or a galvanic skin response (GSR) procedure is well-known. Such contemporary electrodes provide resistive coupling to the test subject, so as to facilitate the monitoring of electrical activity therein.

Although such contemporary resistively coupled electrodes are generally suitable for the intended purposes, resistively coupled electrodes do possess inherent deficiencies which detract from their utility. For example, conductive gels, paste or adhesives are typically utilized when performing an EEG, EMG, EKG or GSR procedure so as to assure the necessary ohmic conduction, i.e., good electrical contact, between such contemporary electrodes and the test subject. The conductive gel, paste or adhesive is generally applied to the contemporary electrode and/or test subject to eliminate non-conductive air gaps there between.

Those skilled in the art will appreciate that the use of such conductive paste, gel and/or adhesive can be very messy, particularly when the test subject has thick hair at the site where the electrode is to be placed. The presence of such hair may necessitate shaving of the site in order to assure adequate electrical contact between the electrode and the skin. The presence of even a very small gap between the contemporary electrode and the surface of the skin, such as that which may be caused by hair, tends to adversely affect the monitoring of electrical activity and is therefore undesirable.

For example, it is common practice for EEG or neurofeedback practitioners to ensure that the resistance of the skin of the test subject's scalp is less than 5k ohms before proceeding with an EEG procedure. In order to obtain such low skin resistance upon the scalp, the neurofeedback practitioner must often utilize an abrasive paste with which the skin of the scalp is rubbed quite intensely. As one may imagine, such intense abrasion of the scalp may cause undesirable pain and may even result in bleeding.

Because of the possible pain and lengthy skin preparation process involved in such EEG procedures, a test subject may postpone or even cancel EEG procedures and may even choose to forego further EEG assessment all together.

The use of such contemporary conductively coupled electrodes may necessitate that the head of the test subject be shaved when, for example, it is necessary to access damage caused by a head injury or brain tumor. During neurofeedback and/or sleep studies, the test subject may be required to wear a helmet or cap within which contemporary conductively coupled electrodes are mounted. Such helmets or caps help to ensure the stability of the position of the conductive electrodes when the electrodes must remain in place for an extended period of time. When such a helmet or cap is utilized, then the neurofeedback practitioner is required to inject a conducting gel or paste through the helmet or cap utilizing a syringe. Occasionally, the neurofeedback or EEG recording practitioner can not obtain good conduction at a particular site such as excessive conducting gel from one site running together with gel from another site and the helmet or cap must be removed so that the problem affecting such conduction may be addressed.

Such repeated application and removal of the helmet is undesirable and time consuming.

The performance and reliability of such contemporary conductively coupled electrodes is degraded by the presence of hair, as well as any other foreign substances (dried blood, dirt, etc.), which might be present upon the skin at the desired sight of the electrode. This is a particular problem when a patient in an emergency room, for example, is suspected of being in cardiac arrest and the doctor needs to perform an EKG measurement as soon as possible.

Hair and other such foreign matter is particularly troublesome in emergency situations, where it may not be possible to shave or clean the affected area. For example, a portable EKG monitor, which may be used to provide medical information to medical personnel at the remote site or may be used to control a defibrilator, must be operated immediately, i.e., without time to shave or clean the sites where electrodes are to be applied to the test subject.

The performance of such a contemporary electrode is degraded by the presence of hair and other materials because hair and other materials tend to physically separate the electrode from the test subject's skin, thereby increasing the resistance of the coupling and degrading the electrical contact between the electrode and the test subject. It is possible that such hair and other material may interfere with the performance of the electrodes sufficiently to render the electrode ineffective in performing its desired function.

In view of the foregoing, it is desirable to provide an electrode suitable for use in EEG, EMG, EKG, and GSR procedures and the like and which does not require conductive coupling to the test subject and is therefore not substantially sensitive to the presence of hair and/or other materials which degrade the performance of contemporary conductively coupled electrodes.

### SUMMARY OF THE INVENTION

The present invention specifically addresses and alleviates the above-mentioned deficiencies associated with the prior art. More particularly, the present invention comprises an electrical activity sensor which comprises an electrode configured to be capacitively coupled to an object being monitored and a variable capacitance coupled to the electrode. The capacitively coupled electrode and the variable capacitance cooperate to mitigate the prior art need for conductively coupling the electrode to the test subject.

The electrode of the present invention comprises a conductive member and a dielectric member which is configured to inhibit contact of the conductive member with the test subject. The conductive member is preferably configured as a disk and the dielectric cover preferably substantially surrounds the disk- shaped conductive member.

The electrode is configured to be capacitively coupled to living tissue. Further, the electrode is preferably configured to be capacitively coupled to a mammal, such as a human being. Those skilled in the art will appreciate that the capacitively coupled electrode of the present invention is suitable for use in various different applications, such as veterinary applications. Indeed, the capacitively coupled electrode of the present invention may be utilized to monitor electrical activity at the surface of non-living or non-biological material.

According to one preferred embodiment of the present invention, the electrode comprises a copper member generally configured as a disk, a dielectric cover substantially surrounding the conductive member and a cap comprised of an insulator which cooperates with a dielectric cover to generally enclose the copper member. At least one conductive lead is coupled to the copper member and extends through the cap, so as to facilitate electrical communication of the electrode with support circuitry, as discussed in detail below.

According to one aspect of the present invention, the variable capacitance comprises an electro-mechanical device. For example, the variable capacitance may comprise at least two spaced apart conductors or plates which define a capacitor and a position controller for varying a position of the two-spaced apart conductors with respect to one another. As those skilled in the art will appreciate, as the two-spaced apart conductors are moved closer to one another, the capacitance of the capacitor defined thereby increases and when the two-spaced apart conductors are moved farther apart from one another, then the capacitance of the capacitor defined thereby decreases.

According to one preferred embodiment of the present invention, a piezoelectric element is disposed intermediate two-spaced apart conductive plates, such that the application of voltage to the piezoelectric crystal effects movement of the two-spaced apart conductive plates, thus varying the capacitance of the capacitor defined thereby. A frequency source, such as a frequency generator, may be utilized to provide electric voltage across the piezoelectric element disposed immediately to the two spaced apart conductive plates. Thus, the frequency source is electrically coupled to the piezoelectric element such that application of the voltage to the piezoelectric element from the frequency source effects movement of the two-spaced apart conductive plates according to well-known electro-mechanical principles.

The frequency source may be configured to provide either a predetermined frequency, e.g., sine wave output, a sequence of different sine outputs, e.g., a sine frequency sweep, or a random frequency output. The random frequency output may comprise either a series of randomly selected sine outputs or white noise like output. Indeed, the output of the frequency source may comprise any desired waveform or sequence of waveforms.

The frequency source preferably comprises a frequency source that is grounded to the living tissue or test item such that the frequency source only affects spacing of the two-spaced apart conductive plates of the variable capacitance and does not otherwise contribute to the output of the electrode. Thus, the frequency source is used only to vary the distance between the two conductive plates.

A detection circuit is coupled to receive an output of the variable capacitor device and to condition this signal so that it is suitable for input to the differential amplifier. Thus, the detection circuit provides a signal which is representative of the input signal at the surface of the living tissue or test item. The detection circuit may, for example, merely comprise a calibrated resistance. The detection circuit conditions the output of the capacitively coupled electrode such that the output of the conductively coupled electrode is suitable for input to a differential amplifier. Thus, the detection circuit provides a signal which is representative of the output of the conductively coupled electrode to the differential amplifier, as discussed in detail below.

An amplifier is coupled so as to amplify an output of the detection circuit. The amplifier preferably comprises a differential amplifier, preferably a variable gain differential amplifier. The differential amplifier has two type of gains: a frequency dependent gain to adjust for the frequency dependent attenuation of the electrode system; and an adjustable frequency independent gain to ensure that the output signal simulate the input signal from the test item. In this manner, adjustments may be made as to compensate for inconsistencies in the electrical components of the electrode system of the present invention, as well as in the efficiency of coupling of the electrode to the test subject. Further, the variable gain amplifier may be adjusted as to amplify the output of the detection circuit in a manner which facilitates provision of an output which generally mimics an output of an EEG electrode, an EKG electrode, an EMG electrode, or a GSR electrode.

An output circuit is coupled to the amplifier so as to define an output impedance. The output impedance may be selected so as to generally mimic the output impedance of an EEG electrode, an EMG electrode, an EKG electrode or a GSR electrode.

The capacitively coupled electrode system of the present invention further comprises a reference electrode which provides a reference to the detection circuit. The capacitively coupled electrode system of the present invention further comprises a ground electrode coupled to an electrically conductive box designed to enclose the electrical components comprising the capacitively coupled electrode of this invention as explained in details below. The reference electrode and the ground electrode function in a manner analogous to reference and ground electrodes of contemporary EEG, EMG, EKG and/or GSR systems.

Thus, according to the present invention, an electrical activity sensor comprising a capacitively coupled electrode electrically coupled to a variable capacitance device utilizes displacement current to sense electrical activity at the surface of a test subject.

These, as well as other advantages of the present invention, will be more apparent from the following description and the drawings. It is understood that changes in the specific structure shown and described may be made within the scope of the claims without departing from the spirit of the invention.

### DESCRIPTION OF THE DRAWINGS

FIG. 1 is a block diagram showing the system for sensing and reproducing electrical signals according to the present invention;

FIGS. 2A and 2B show one example of a capacitively coupled electrode formed according to the present invention;

FIG. 3 shows one example of a variable capacitance device formed according to the present invention.

FIG. 4 is a simplified electrical schematic (as used in a circuit simulation) showing the system for sensing and reproducing electrical signals according to the present invention;

FIG. 5 is a graph showing an exemplary input signal, (V_{IN}) of FIG. 4, as used in a simulation of the present invention;

FIG. 6 is a graph showing an exemplary output voltage, (V_{OUT})of FIG. 4, according to the simulation; and

FIG. 7 is a Bode diagram showing output voltage and phase versus frequency according to the simulation of the circuit of FIG. 4.

### DETAILED DESCRIPTION OF THE INVENTION

The detailed description set forth below in connection with the appended drawings is intended as a description of the presently preferred embodiment of the invention and is not intended to represent the only form which the present invention may be constructed or utilized. The description sets forth the functions and the sequence of steps for constructing and operating the invention in connection with the illustrated embodiment. It is to be understood, however, that the same or equivalent functions and sequences may be accomplished by different embodiments that are also intended to be encompassed within the spirit and scope of the invention.

The present invention is generally described herein as being particularly suited for use in medical applications such as an electroencephalograph (EEG), an electromyograph (EMG), an electrocardiograph (EKG) or a galvanic skin response (GSR) device. However, such description is by way of illustration only, and not by way of limitation. Indeed, the present invention may find applications in various unrelated fields. Thus, the present invention may be utilized to capacitively couple an electrode to any desired test items, either living, dead, inanimate, organic or inorganic. Indeed, the present invention may be utilized to measure electrical activity in any desired test item for which such capacitive coupling is appropriate.

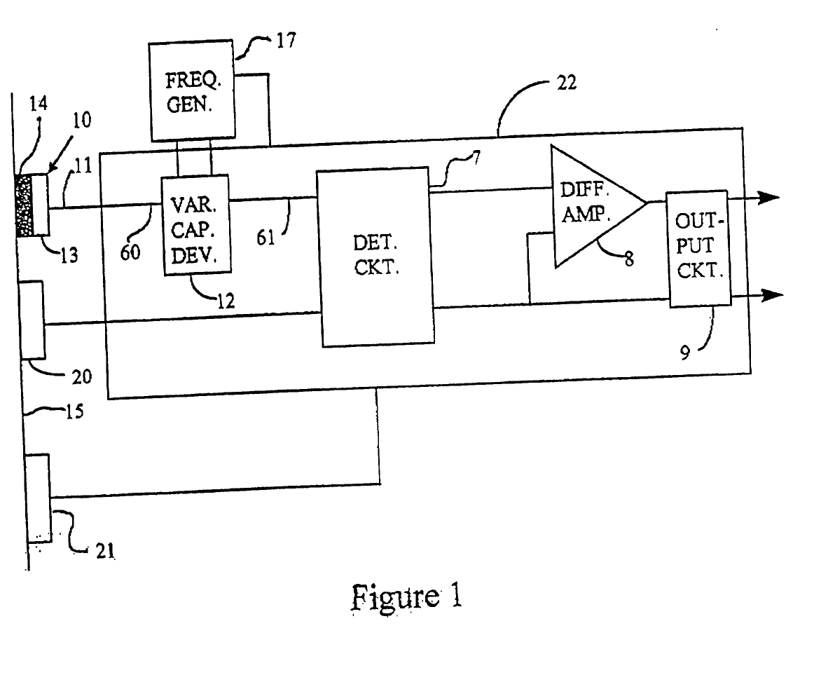

Referring now to FIG. 1, an exemplary embodiment of the capacitively coupled electrode system of the present invention generally comprises a capacitively coupled electrode 10 which is in electrical communication with a variable capacitance device 12. A detection circuit 7 receives the output of the variable capacitance device 12 and conditions the output of the variable capacitance device 12 as described below. An amplifier 8 receives the output of the detection circuit 7 and amplifies or attenuates that output as described below. An output circuit 9 is in electrical communication with the amplifier 8 so as to receive the output of the amplifier 8 and determine an output impedance of the capacitively coupled electrode system.

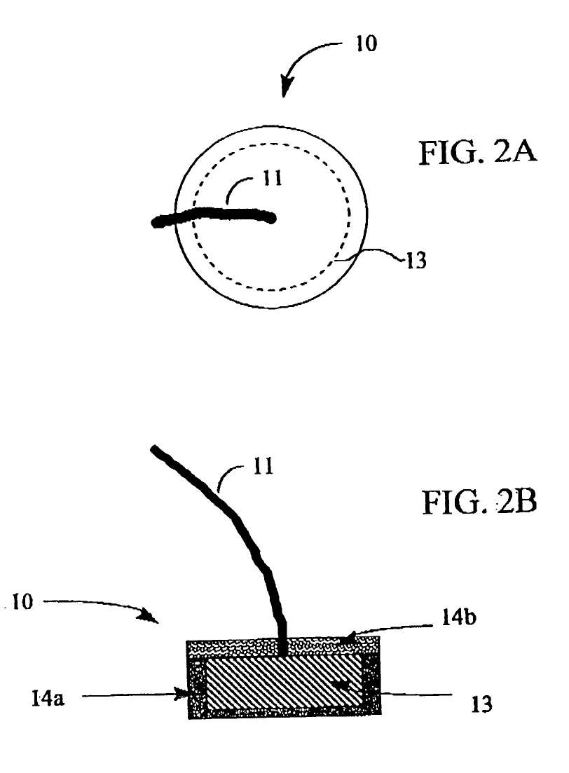

The capacitively coupled electrode 10 (better shown in FIGS. 2A and 2B) generally comprises a conductive member 13 and a non-conductive member 14. The conductive member 13 defines a capacitor plate which facilitates the sensing of electrical activity within a test item or subject 15. The non-conductive member 14 electrically isolates the conductive member 13 from the test subject 15.

Thus, the capacitively coupled electrode 10 is capacitively coupled, rather than conductively coupled, to the test subject 15. Because of this capacitive coupling, displacement current may be utilized to effect sensing of electrical signals at the test subject. As discussed above, such capacitive coupling provides substantial advantages in eliminating the need for good electrical contact between the electrode 10 and the test subject 15.

Various different configurations of the capacitively coupled electrode 10 are contemplated. For example, the conductive member 13 of the capacitively coupled electrode 10 may be electrically isolated from the test subject 15 via a non-conducting layer 14 formed upon one surface thereof only, as shown in FIG. 1. Alternatively, the conductive member 13 of the capacitively coupled electrode 10 may be substantially encapsulated within a non-conductor as shown in FIGS. 2A and 2B. Substantially encapsulating the conductive member 13 within a non-conducting layer 14 mitigates the likelihood of the conductive member 13 inadvertently contacting the test subject, and thus degrading the performance of the capacitively coupled electrode of the present invention.

The variable capacitance device 12 is generally defined by a capacitor, the capacitance of which can be varied, preferably in a controlled fashion. Thus, the plate area of the capacitor, the spacing between the plates of the capacitor and/or the dielectric constant of the capacitor of the variable capacitance device 12 may be varied. According to the preferred embodiment of the present invention, a frequency source 17 provides a frequency input to the variable capacitance device 12, so as to effect varying of the capacitance of the variable capacitance device 12, as desired. The detection circuit 7 conditions the output of the variable capacitance device 12, so as to make the signal suitable for amplification by the amplifier 8.

The frequency generator may comprise a commercially available frequency generator or, alternatively, may comprise a frequency generator built specifically for use with the variable capacitance device 12. In either instance, the frequency source 17 is preferably electrically grounded to the electrical box 22 to provide protection to the remainder of the capacitively coupled electrode system, so as to mitigate any likelihood of an undesirable electrical shock to the patient.

The frequency generator 17 may optionally be disposed within the box depending on its size. In case it is out of the box 22, the frequency generator 17 should be grounded to the box 22. The role of the ground electrode 21 connected to the box 22 is to protect the test item from any possible electrical shocks that could be generated by the electrical components of the electrode circuit. This type of grounding using a box with electronic components inside it to protect a test item from possible electric shocks is standard procedure in the industry of EEG systems.

Referring now to FIGS. 2A and 2B, an exemplary capacitively coupled electrode is shown. With particular reference to 2A, the exemplary capacitively coupled electrode 10 is preferably generally circular in configuration, so as to define a disk. However, those skilled in the art will appreciate that various other configurations of the capacitively coupled electrode 10 are likewise suitable. A conductive conduit or lead 11 extends from the capacitively coupled electrode 10 so as to facilitate electrical communication with the variable capacitance device 12 (FIG. 1). Lead 11 is electrically coupled to the conductive member 13 of the capacitively coupled electrode 10.

With particular reference to FIG. 2B, the conductive member 13 of the capacitively coupled electrode 10 may, if desired, be generally completely encapsulated within a non-conductive housing so as to mitigate problems associated with inadvertent contact of the conductive member 13 with the test subject 15 (FIG. 1). As shown in FIG. 2B, a dielectric material contacting portion 14A generally surrounds most of the conductive member 13 and a dielectric cap 14B generally covers the remaining portion of the conductive member 13. The lead 11 is insulated. Thus, inadvertent electrical contact with the test subject of the lead 11 and/or the conductive member 13 is substantially inhibited.

The conductive member 13 is preferably comprised of copper. However, those skilled in the art will appreciate that various other conductive substances, particularly metals, are likewise suitable. The non-conductive housing 14A, 14B, may be comprised of any suitable, preferably biologically compatible, dielectric material such as plastic, rubber, epoxy, etc.

The conductive member 13 is preferably about 1 cm diameter but the dimension can be changed to fit the needs of the clinical or other setting. The shape of the electrode can also be varied as desired. Thus, the electrode can be sized and configured so as to be suitable for the test item or test subject. The wire or lead 11 itself is preferably a part of the electrode of this invention. The front side of the electrode (the active side, which is the side in contact with the body or almost in contact with the body if there is something preventing direct contact, such as body hairs) is covered with a thin layer of a material with a high dielectric constant such as Teflon or a ceramic. Such materials have a high dielectric constant, which is ideal for this application. The backside of the electrode is protected by an insulating material.

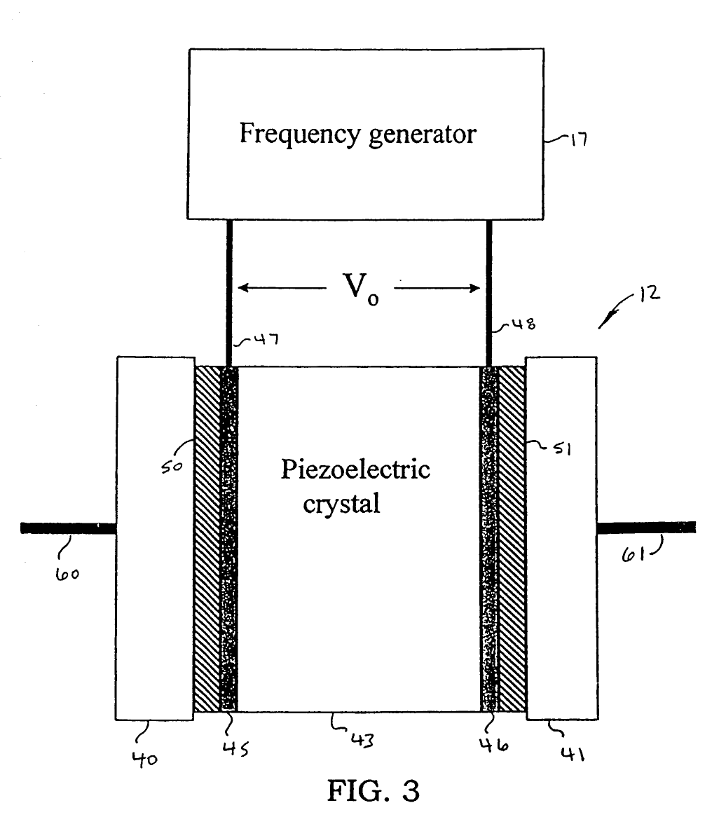

Referring now to FIG. 3, an exemplary embodiment of the variable capacitance device 12 comprises first 40 and second 41 conductive plates which define a capacitor. The first 40 and second 41 conductive plates are movable with respect to one another, such that the distance there between is easily varied. A piezoelectric crystal 43 or the like is disposed intermediate the first 40 and second 41 conductive plates so as to affect movement of the first 40 and second 41 conductive plates relative to one another. The frequency source 17 is coupled so as to provide a voltage across the piezoelectric crystal 43 in order to effect compression and expansion of the piezoelectric crystal 43, thus varying the distance between the first 40 and second 41 plates of the capacitor defined thereby. In this manner, the frequency source 17 controls the capacitance of the variable capacitance device 12.

Preferably, conductive coatings 45 and 46 are applied to the piezoelectric crystal 43, so as to facilitate desired electrical contact with the leads 47 and 48, which provide electrical communication between the piezoelectric crystal 43 and the frequency source 17.

Preferably, epoxy layers 50 and 51 facilitate mechanical attachment of the piezoelectric crystal 43 (via the conductive coatings 45 and 46) to the conductive plates 40 and 41. Those skilled in the art will appreciate that various other means for fastening the conductive plates 40 and 41 to the piezoelectric crystal are likewise suitable. For example, the conductive plates 40 and 41 may be held in place with respect to the piezoelectric crystal 43 via the use of fasteners such as screws, preferably in combination with spring washers, such as Belville washers, which pass through the conductive plates 40 and 41 and the piezoelectric crystal 43. As a further alternative, spring clips may be utilized to bias the conductive plates 40 and 41 toward the piezoelectric crystal 43.

Lead 60 facilitates electrical communication of the first plate 40 with the capacitively coupled electrode 10. Similarly, lead 61 facilitates electrical communication of the second plate 41 with the detection circuit 7.

The variable frequency source 17, such as a commercially available frequency generator, generates a sinusoidal voltage V_{O}=V₀′ sin ω′t. This voltage is applied to a piezoelectric crystal 43 placed between the two plates 40 and 41 of the parallel plate variable capacitor. The voltage V_{O} is transmitted to the crystal 43 through conduction plates 45 and 46, which cover the side surfaces of the piezoelectric crystal 43. The piezoelectric crystal 43 is attached to the two plates in such a manner that the voltage V₀ cannot leak to the parallel plates 40 and 41 of the variable capacitor 12 (in which case this voltage V₀ would interfere with the potential of the body). This is preferably accomplished by attaching the crystal of the plates 40 and 41 using an epoxy having a high dielectric constant. The applied voltage V₀ modify the thickness of the piezoelectric crystal in a sinusoidal manner. This results in a sinusoidal modulation of the distance between the plates of the capacitor d=d₀(1+δ sin ω′t) where dₒ is the distance between the two plates of the parallel plate capacitor when there is no voltage applied to the piezoelectric crystal, i.e., V₀=0. The parameter δ is a modulation factor dependent in a complex manner on the amplitude V₀′ of the applied voltage. The resulting modulation of the capacitance is C=C′/(1+δ sin ω′t) with C′=κεₒA/dₒ. In the latter equation, κ is the average value of the dielectric constant of the materials between the plates (κ=1 for air), εₒ is the permittivity constant of the vacuum and A is the surface of one of the plates of the parallel plate capacitor.

The active capacitively coupled electrode with the variable capacitance of this invention can be secured to a living body in many different ways depending on the application. For EEG measurements, the best way to secure the electrodes in place on the scalp is to use a helmet. The electrodes can be fixed tightly in holes corresponding to the exact location of the locations described in the 10-20 international system of EEG electrode placement. Monitoring EMG activity on a limb can be done using a stretch band stretching around the limb. The extremities of the band could be fixed together using the Velcro system. The same procedure using stretch bands can be used on the torso for EKG measurements, for example. In these cases, the electrode would be embedded in the tissue of the stretching band. Other methods of fixing the electrodes could include the use of tape or adhesives (on the limbs or the main body), using a holder arm firmly fixed to the patients' bed or chair or other furniture around her/him, etc.

In operation, a frequency source 17 provides a predetermined frequency, a sequence of predetermined frequencies or random frequencies which excite the piezoelectric crystal 43 so as to effect vibration of the piezoelectric crystal 43. Vibration of the piezoelectric crystal 43 varies the spacing of the first 40 and second 41 plates of the variable capacitance device 12.

Further according to one embodiment of the present invention, the detection circuit 7 merely comprises a resistor which develops a voltage drop across the two inputs to the amplifier 8.

The detection circuit 7 is in electrical communication with a reference electrode 20. The reference electrode 20 and/or the ground electrode 21 are preferably contemporary conductively coupled electrodes and are preferably coupled to a monitoring device such as an EEG monitor, an EMG monitor, an EKG monitor or a GSR monitor according to well-known principles. Alternatively, the reference electrode 20 and the ground electrode 21 are capacitively coupled electrodes formed according to the present invention and are coupled to the monitoring device in a manner analogous to coupling of the capacitively coupled electrode 10 thereto.

When used in the performance of an EEG, for example, then the reference electrode 20 is typically attached to a patient at a location close to the location of the capacitively coupled electrode 10, such as at the lobe of one ear. During EEG procedures the ground electrode is typically placed on the patient in a region of lowest electrical potential, such as a boney structure, typically the boney structure of the C-7 vertebra.

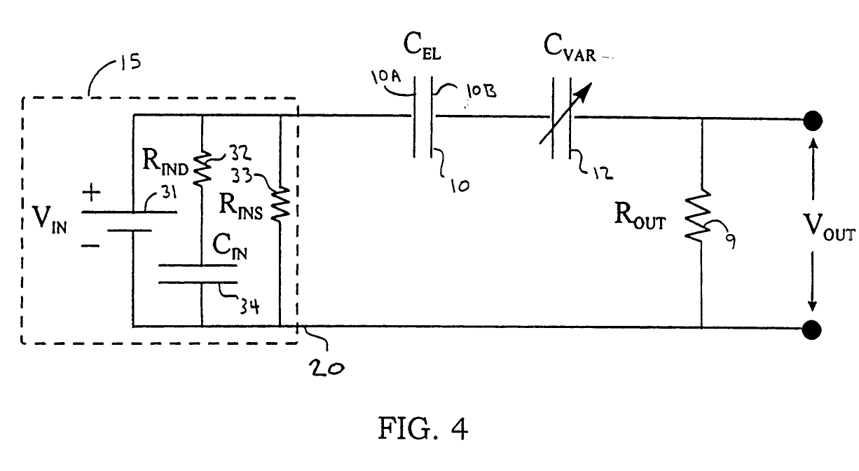

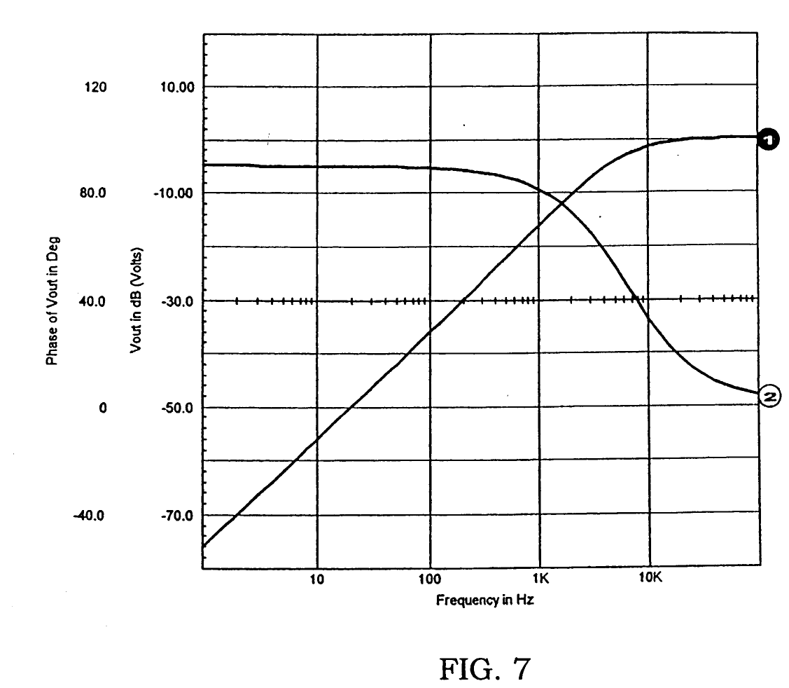

The amplifier 8 preferably comprises a variable gain differential amplifier, so as to facilitate adjustment of the amplitude of the signal output hereby. The variable gain differential amplifier provides a frequency dependent gain adjustment as a compensation for the frequency dependent transfer function of the electrode system as shown in the Bode diagram of FIG. 7. FIG. 7 shows a logarithmic dependence of the output voltage (Vₒᵤₜ in FIG. 4) with the frequency f of the input signal of the test item at low frequencies (f<10 kHz). This dependence is compensated by an inverse logarithmic dependence of the amplifier gain to be adjusted to the specific condition of each capacitive electrode of this invention. Additionally, the differential amplifier has a general gain to adjust the overall output voltage to match exactly the amplitude of the input voltage of the test item. Adjustment of the output of the amplifier 8 facilitates use of the capacitively coupled electrode system of the present invention in a variety of different applications, including but not limited to EEG, EMG, EKG and GSR applications. As those skilled in the art will appreciate, the electrodes utilized in each of these different procedures are generally different from one another, and therefore generally provide different output amplitudes. Thus, by adjusting the amplifier 8, an amplitude which is generally representative of the desired electrode, e.g., EEG electrode, EMG electrode, EKG electrode or a GSR electrode, can be provided.

Referring now to FIG. 4, a simplified schematic of the present invention shows the basic components thereof cooperating with a test subject is to provide an output signal (V_{OUT}). This simplified electrical schematic was used in a simulation to validate the desired operation of the present invention.

The test subject 15 is simulated with: a voltage source 31; a resistor 32 in series with a capacitor 34, both of which are in parallel with the voltage source 31; and a resistor 33 which is also in parallel with the voltage source 31. The voltage source 31 provides a varying input voltage V_{IN}. The resistor 32 has a resistance R_{IND}. The capacitor 34 has a capacitance C_{IN}. The resistor 33 has a resistance R_{INS}.

The capacitively coupled electrode 10, in combination with the test subject 15, defines a capacitor which provides a capacitance C_{EL}. That is, the test subject 15 defines a first plate 10A of the capacitor and the capacitively coupled electrode 10 defines the second plate 10B thereof. In this manner, electrical activity within the test subject 15 is sensed as displacement current through the closed loop circuit formed by the subject's equivalent circuit and C_{EL}, C_{VAR} and R_{OUT} Variable capacitance 12 provides a varying capacitance Cᵥₐᵣ. Output resistor 9 provides an output resistance ROUT and is capacitively coupled with the test subject 15 via capacitively coupled electrode 10 and variable capacitance device 12 on one side thereof and is conductively coupled to the test subject 15 on the other side thereof via the reference electrode 20.

It can be seen that a closed loop circuit is formed by the test subject 15, the capacitively coupled electrode 10, the variable capacitance device 12, the resistor 9 and the reference electrode 20. If the variable capacitance device 12 is considered to be simply a parallel plate capacitor whose capacitance Cᵥₐᵣ is changed by a fast sinusoidal variation of the distance d between the capacitor plates such that d=dₒ(1+δ sin(ω′t)), then Cᵥₐᵣ=Cᵥₐᵣ′/(1+δ sin(ω′t)) with Cᵥₐᵣ′=ε₀A/d. In the last two equations, dₒ is the distance between the two plates of the parallel plate capacitor at t=0 second, δ is the fraction of modulation of the capacitance of the variable capacitor (δ=1 represents 100% modulation; δ=0 represents no modulation), ω′=2πf′ with f′ the frequency of variation of the distance between the capacitor plates, εₒ is the permittivity of a vacuum and A is the surface of one plate of the parallel plate capacitor.

Assuming that the detection circuit is a simple resistor, the closed loop circuit can be readily analyzed to give the voltage output V_{OUT} to be fed to the variable differential amplifier. The resulting circuit is presented in FIG. 4, along with the symbols representing the variables used in the mathematical analysis. For the purpose of this analysis, the living body is modeled as a skin surface resistor R_{INS} in parallel with a low frequency voltage source V_{IN} both in parallel with a capacitor C_{IN} in series with a dermis resistor R_{IND}.

The definition of the variables in FIG. 4 is as follows: V_{IN}=V sin ωt is the slowly varying voltage generated by the body between the capacitively coupled electrode and the reference electrode; R_{INS} is the electrical resistance of the epidermis between the capacitively coupled electrode and the reference electrode at the surface of the skin; C_{IN} is the capacitance of the body between the capacitively coupled electrode and the reference electrode mainly generated at the basal membrane (between the epidermis and the dermis); R_{IND} is the electrical resistance of the epidermis and dermis regions in series with C_{IN}; C_{EL} is the capacitance of the capacitively couple electrode; C_{VAR} is the capacitance of the variable capacitor; and R_{OUT} is the resistance of the detection resistor.

If the circuit components C_{EL}, C_{VAR} and R_{OUT} are chosen carefully, they can serve as a filter to filter out the high frequency component f′ of the variable capacitor (even if these components look placed to form a high pass filter). The statement will be justified below with the results of the simulations. In that case, one can average the high frequency components of the mathematical analysis and calculate an expression of the output voltage V_{OUT} which depends only on the low frequency f generated by the test item. The resulting formula for the voltage V_{OUT} across the detection resistor R_{OUT} is:

$${V}_{OUT} = V \{ \frac{{ω}^{2} {R}_{OUT}^{2} {C}_{eq}^{2} cos ω t - ω {R}_{OUT} {C}_{eq} sin ω t}{1 + {ω}^{2} {R}_{OUT}^{2} {C}_{eq}^{2}} \} + small correction terms .$$

In the above equation C_{eq}=(C_{VAR}^{′-1}+C_{EL}⁻¹)⁻¹ is the equivalent capacitance, ω=2πf, f is the frequency of oscillation of V_{IN} in cycles per second or Hz, π=3.1416. The equation for V_{OUT} above assumes a sinusoidal variation of the distance between the two plates of a parallel plate capacitor at the frequency f′=ω′/2π which is much larger than f=ω/2π. This sinusoidal variation is just one example of an infinite number of ways the capacitance of the variable capacitor can be varied. For example, the capacitance C_{VAR} could be varied by varying the permittivity of a dielectric material placed between the two plates such that ε=ε₀(1+δ sin ωt). Alternatively, the surface of the plates of C_{VAR} can be varied as A=A₀(1+δ sin ωt). Methods for varying the permittivity ε or the area A of the plates are well-known.

In order to check the validity of the above equation, a simulation of the closed loop circuit analyzed above was performed using a commercially available circuit simulation software. For the simulation purposes, the following parameter values were chosen:

V=2 μV

f′=ω′/2π=10,000 Hz

R_{IND}=1 kΩ

R_{INS=}100 kΩ

C_{IN}=40 nF

C_{EL}=3 pF

C_{VAR}′=1 μF

δ=0.5

f=ω/2π=1 Hz

R_{OUT}10 MEGΩ

These parameters were chosen to simulate an EEG signal at the input and to provide the highest output signal possible without any distortion.

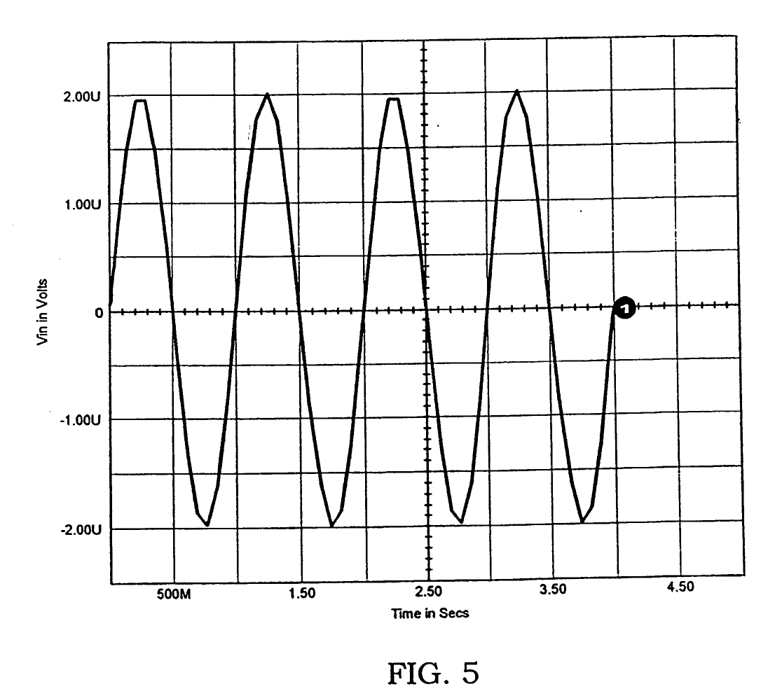

FIG. 5 presents the generally sinusoidal input signal V_{IN}=V sin ωt.

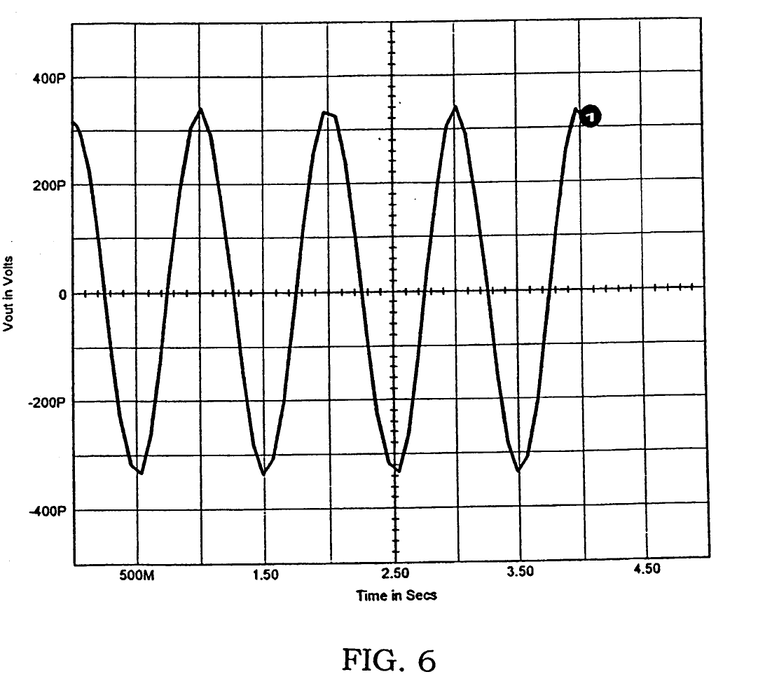

FIG. 6 presents the generally sinusoidal output voltage V_{OUT} . With the values chosen above ωR_{OUT}C_{eq}=1.88×10⁻⁴<<1 and the maximum amplitude of V_{OUT}/V_{OUT}/max 15 is /V_{OUT}/max=VωR_{OUT}c_{eq}=3.77×10⁻¹⁰cos ωt,in a very good agreement with the simulation shown in FIG. 6.

FIG. 7 presents a Bode diagram (output voltage and phase vs. frequency) for the simulation parameters described above. One may note the saturation of the output voltage above f′=10,000 Hz. The equation for V_{OUT} shows that the output voltage should be independent of the frequency of modulation of the capacitor f′ and the fraction of modulation of the capacitance δ. V_{OUT} should also be independent of Cᵥₐᵣ′ as long as Cᵥₐᵣ′>>C_{EL}. The independence of the output voltage on f′ is apparent in FIG. 6, as no high frequency modulation signal is observed. This result justify our assumption to average the high frequency terms that are generated by the variable capacitor as mentioned previously when calculating the output voltage V_{OUT}. Additional simulations showed that there were no change in V_{OUT} for 0.1<δ<0.9 and when Cᵥₐᵣ′>>C_{EL}. More simulations showed that the linear dependency of V_{OUT} on ω, R_{OUT} and C_{eq} is valid as long as ωR_{OUT}C_{eq}<<1 and C_{VAR}′>>C_{EL}.

The presence of the variable capacitance is not only desirable, but is important for the electrode to function as described. The variable capacitance generates the displacement current without which there is no current in the circuit comprised of the electrode, the variable capacitor and the detection circuit. For the clarity of the discussion here, let us call the circuit mentioned in the last sentence the electrode circuit. The electrical potential generated by the test item is generally too weak to generate any current in the electrode circuit (especially in the case of EEG). Without a current in the electrode circuit, there is no means to recover the potential generated by the test item (unless we use resistively coupled electrodes which is what we are trying to avoid with this invention).

The goal of the electrode of this invention is to monitor the electrical potential generated at the surface of the tissue of the test item without distortion and without the use of a resistively coupled electrode. This is accomplished by capacitively coupling the electrode to the test item and by generating a variable current in the electrode circuit.

There are two other ways we know to generate a variable current in the electrode circuit. These are: to include in the electrode circuit a variable voltage source or to include in the electrode circuit a variable current source. There are problems with both methods. The problem with adding a variable voltage source is that this variable voltage is added to the very small potential generated at the surface of the skull (in the case of EEG, for example). To separate these two voltages accurately would require complex electronic circuits because they are so small (in the microvolt range for EEG). The problem with adding a current source is that the voltage at the detection circuit includes an amplitude modulation (AM) of the potential generated by the test item and the voltage generated in the electrode circuit by the variable current source. This is similar to AM modulation used for radio transmission. This would need an AM demodulator, a complex circuit for such a would-be simple electrode. The variable capacitor eliminates these problems.

Electric circuit theory and electrical simulations using a commercially available software showed that if the variable capacitor is varied at a frequency that is at least 10 times the maximum frequency expected to be generated by the test item, then there is a possibility to eliminate the effect of this rapidly varying capacitor simply by choosing the components of the electrode circuit in such a manner that this circuit act like a filter which filter out high frequency components and leave intact the low frequency components generated by the test item. This is the secret of the simplicity of the electrode of this invention and it is due to the use of a variable capacitor and cannot be obtained in any other way we could think of. We hope this clarify the reasons for the use of a variable capacitance.

It is understood that the exemplary capacitively coupled electrode system described herein and shown in the drawings represents only a presently preferred embodiment of the invention. Indeed, various modifications and additions may be made to such embodiment without departing from the spirit and scope of the invention. For example, various different configurations of the electrode and/or variable capacitance device are contemplated. Thus, these and other modifications and additions may be obvious to those skilled in the art and may be implemented to adapt the present invention for use in a variety of different applications.

1. An electrical activity sensor comprising:
an electrode configured to be capacitively coupled to a test item;
a variable capacitance coupled to the electrode; and
wherein the capacitively coupled electrode and the variable capacitance cooperate to mitigate a need for conductively coupling an electrode to the test item.

2. The electrical activity sensor as recited in claim 1, wherein the electrode comprises:
a conductive member; and
a dielectric member configured to inhibit contact of the conductive member with the test item.

3. The electrical activity sensor as recited in claim 1, wherein the electrode comprises:
a conductive member generally configured as a disk; and
a dielectric cover substantially surrounding the conductive member.

4. The electrode system as recited in claim 1, wherein the electrode is configured to be capacitively coupled to living tissue.

5. The electrode system as recited in claim 1, wherein the electrode is configured to be capacitively coupled to a mammal.

6. The electrode system as recited in claim 1, wherein the electrode is configured to be capacitively coupled to a human being.

7. The electrical activity sensor as recited in claim 1, wherein the electrode comprises:
a copper member generally configured as a disk;
a dielectric cover substantially surrounding the conductive member;
a cap comprised of insulator cooperating with the dielectric cover to generally enclose the copper member; and
a conductive lead coupled to the copper member and extending through the cap.

8. The electrical activity sensor as recited in claim 1, wherein the variable capacitance comprises an electro-mechanical device.

9. The electrical activity sensor as recited in claim 1, wherein the variable capacitance comprises:
at least two spaced apart conductors; and
a position controller for varying a position of the conductors with respect to one another.

10. The electrical activity sensor as recited in claim 1, wherein the variable capacitance comprises:
two spaced apart conductive plates; and
a piezoelectric element disposed intermediate the two spaced apart conductive plates such that application of a voltage to the piezoelectric crystal effects movement of the two spaced apart conductive plates.

11. The electrical activity sensor as recited in claim 1, wherein the variable capacitance comprises:
a frequency source;
two spaced apart conductive plates; and
a piezoelectric element disposed intermediate the two spaced apart conductive plates and coupled to the frequency source such that application of a voltage to the piezoelectric element from the frequency source effects movement of the two spaced apart conductive plates.

12. The electrical activity sensor as recited in claim 1, wherein the variable capacitance comprises:
a frequency source configured to provide a generally predetermined frequency output;
two spaced apart conductive plates; and
a piezoelectric element disposed intermediate the two spaced apart conductive plates and coupled to the frequency source such that application of a voltage to the piezoelectric crystal from the frequency source effects movement of the two spaced apart conductive plates.

13. The electrical activity sensor as recited in claim 1, wherein the variable capacitance comprises:
a frequency source configured to provide a generally random frequency output;
two spaced apart conductive plates; and
a piezoelectric element disposed intermediate the two spaced apart conductive plates and coupled to the frequency source such that application of a voltage to the piezoelectric crystal from the frequency source effects movement of the two spaced apart conductive plates.

14. The electrical activity sensor as recited in claim 1, wherein the variable capacitance comprises:
frequency source grounded to a metal enclosure;
two spaced apart conductive plates; and
a piezoelectric element disposed intermediate the two spaced apart conductive plates and coupled to the frequency source such that application of a voltage to the piezoelectric crystal from the frequency source effects movement of the two spaced apart conductive plates.

15. The electrical activity sensor as recited in claim 1, further comprising a detection circuit coupled to receive an output of the capacitively coupled electrode and to condition the output of the capacitively coupled electrode.

16. The electrical activity sensor as recited in claim 1, further comprising a detection circuit coupled to receive an output of the capacitively coupled electrode, the detection circuit comprising a calibrated resistance.

17. The electrical activity sensor as recited in claim 1, wherein further comprising a detection circuit coupled to receive an output of the capacitively coupled electrode, the detection circuit being configured so as to provide an output suitable for input to a differential amplifier.

18. The electrical activity sensor as recited in claim 1, further comprising:
a detection circuit coupled to condition an output of the capacitively coupled electrode; and
an amplifier coupled to amplify an output of the detection circuit.

19. The electrical activity sensor as recited in claim 1, further comprising:
a detection circuit coupled to condition an output of the capacitively coupled electrode; and
a differential amplifier coupled to amplify an output of the detection circuit.

20. The electrical activity sensor as recited in claim 1, further comprising:
a detection circuit coupled to condition an output of the capacitively coupled electrode; and
a variable gain amplifier coupled to amplify an output of the detection circuit.

21. The electrical activity sensor as recited in claim 1, further comprising:
a detection circuit coupled to condition an output of the capacitively coupled electrode; and
a variable gain amplifier coupled to amplify an output of the detection circuit in a manner which facilitates provision of an output that generally mimics an output of at least one of an electroencephalograph electrode, an electrocardiograph electrode, an electromyograph electrode and a galvanic skin response electrode.

22. The electrical activity sensor as recited in claim 1, further comprising:
a detection circuit coupled to condition an output of the capacitively coupled electrode;
an amplifier coupled to amplify an output of the detection circuit; and
an output circuit coupled to the amplifier to define an output impedance.

23. The electrical activity sensor as recited in claim 1, further comprising:
a detection circuit coupled to condition an output of the capacitively coupled electrode;
an amplifier coupled to amplify an output of the detection circuit; and
an output circuit coupled to the amplifier to define an output impedance which is suitable for providing a signal to an electroencephalograph.

24. The electrical activity sensor as recited in claim 1, further comprising:
a detection circuit coupled to condition an output of the capacitively coupled electrode;
an amplifier coupled to amplify an output of the detection circuit; and
an output circuit coupled to the amplifier to define an output impedance which is suitable for providing a signal to an electromyograph.

25. The electrical activity sensor as recited in claim 1, further comprising:
a detection circuit coupled to condition an output of the capacitively coupled electrode;
an amplifier coupled to amplify an output of the detection circuit; and
an output circuit coupled to the amplifier to define an output impedance which is suitable for providing a signal to an electrocardiograph.

26. The electrical activity sensor as recited in claim 1, further comprising:
a detection circuit coupled to condition an output of the capacitively coupled electrode;
an amplifier coupled to amplify an output of the detection circuit; and
an output circuit coupled to the amplifier to define an output impedance which is suitable for providing a signal to a galvanic skin response monitor.

27. The electrical activity sensor as recited in claim 1, further comprising a reference electrode coupled to the detection circuit.

28. The electrical activity sensor as recited in claim 1, further comprising a ground electrode coupled to a metal enclosure.

29. The electrical activity sensor as recited in claim 1, further comprising a reference electrode coupled to the detection circuit and a ground electrode coupled to a metal enclosure.

30. An electrical activity sensor comprising an electrode coupled to a variable capacitance device.

31. A method for characterizing electrical activity of an object being monitored, the method comprising using displacement current to sense electrical activity within a test item.

32. The method as recited in claim 31, wherein using displacement current to sense electrical activity comprises capacitively coupling an electrode to the object being tested.

33. The method as recited in claim 31, wherein using displacement current to sense electrical activity comprises capacitively coupling an electrode to the object being tested and varying a capacitance of a capacitor coupled to the electrode.

34. The method as recited in claim 31, wherein using displacement current to sense electrical activity comprises capacitively coupling an electrode to the object being tested and using a frequency source to vary a capacitance of a capacitor coupled to the electrode.

35. The method as recited in claim 31, wherein using displacement current to sense electrical activity comprises capacitively coupling an electrode to the object being tested and using a frequency source to vary a capacitance of a capacitor coupled to the electrode, the capacitance of the capacitor being varied in a predetermined manner.

36. The method as recited in claim 31, wherein using displacement current to sense electrical activity comprises capacitively coupling an electrode to the object being tested and using a frequency source to vary a capacitance of a capacitor coupled to the electrode, the capacitance of the capacitor being varied in a random manner.
